(19) United States
(12) Patent Application Publication
(10) Pub. No.: US 2017/0273782 A1
(43) Pub. Date: Sep. 28, 2017

(54) CARDIAC VALVE SUPPORT DEVICES HAVING IMPROVED COMPATIBILITY WITH TRANSCATHETER PROSTHETIC VALVES

(71) Applicant: MVALVE TECHNOLOGIES LTD., Herzliya (IL)

(72) Inventors: Amit TUBISHEVITZ, Tel Aviv (IL); Shay DUBI, Tel Aviv (IL)

(73) Assignee: MVALVE TECHNOLOGIES LTD., Herzliya (IL)

(21) Appl. No.: 15/505,392

(22) PCT Filed: Aug. 18, 2015
(86) PCT No.: PCT/IL2015/050836

§ 371 Date: Feb. 21, 2017

Related U.S. Application Data
(60) Provisional application No. 62/039,937, filed on Aug. 21, 2014.

(51) Int. Cl. A61F 2/24

(52) U.S. Cl. CPC A61F 2/2409; A61F 2/243; A61F 2250/006; A61F 2/2412

(57) ABSTRACT

The present invention is primarily directed to a valve support device suitable for endovascular and/or transapical implantation at or near to a cardiac valve annulus, comprising one or more support rings and one or more height-increasing elements attached to the inner circumference of said support rings. Preferred embodiments of height-increasing elements for use in the present invention include wire springs of various types, fabric sleeves and tab-like elements.

### FIELD OF THE INVENTION

The present invention relates to support devices for replacement cardiac valves having improved compatibility with prosthetic cardiac valves that are intended for transcatheter delivery. More specifically, the devices of the present invention are cardiac valve support devices comprising one or more height-increasing elements.

### BACKGROUND OF THE INVENTION

The present inventors have previously disclosed valve support devices which, following their implantation at a cardiac valve annulus, are intended to provide a stable landing platform for the subsequent or concurrent implantation of a commercially-available prosthetic heart valve. These valve support devices generally have the form of a single flattened ring (as described in WO 2013/128436) or two such rings interconnected by bridging elements (as disclosed in US 2014/0005778). However, most of the prosthetic heart valves intended for transcatheter delivery that are in current use are designed to be implanted in a tube-like area within the body—for example within the aorta. Consequently, in many cases, the prosthetic valve itself has a tubular structure. As a result, the implantation of such valves within a ring-like valve support device element may be more difficult, since in the absence of the aforementioned tube-like structure, the prosthetic valve may require more accurate positioning thereby extending the length of the cardiac procedure, and may furthermore lead to excessive wear of the valve.

The present inventors have overcome the aforementioned problems associated with the implantation of prosthetic valves—and particularly, of valves having an essentially tubular form—within a ring-like valve support device, by means of incorporating certain additional features into the support device.

### SUMMARY OF THE INVENTION

The present invention is primarily directed to a valve support device suitable for endovascular and/or transapical implantation at or near to a cardiac valve annulus, wherein said device comprises one or more support rings, and one or more height-increasing elements attached to the inner circumference of said one or more said support rings. The advantages of the height-increasing elements stem from the fact that in essence they transform the inner portion of the flat ring-shaped valve support device into a tube-like shape, thus better mimicking the form of the prosthetic valves (since both the aortic valve and the aorta have an essentially tubular shape).

In one preferred embodiment of the invention, the aforementioned height-increasing element is provided by a wire spring, manufactured from a biocompatible metal such as Nitinol. In this embodiment, the lower border of said spring may be attached to the inner circumference of the support ring(s) such that said spring is located above the plane of said ring(s) (i.e. towards the atrium).

In an alternative version of this embodiment, the upper border of said spring may be attached to the inner circumference of the support ring(s), such that said spring is located below the plane of said ring(s) (i.e. towards the ventricle).

In a further version of this embodiment, the central portion of the spring is attached to the inner circumference of the support ring(s), such that portions of said spring are disposed both above and below the plane of the support ring(s). Finally, in another version, two separate springs may be present, with one spring disposed above the plane of the support ring, and the second spring disposed below this plane.

Optionally, the wire springs and/or the support ring(s) in any of the above-disclosed embodiments may be covered with a biocompatible fabric.

In a yet further embodiment of the present invention, the height-increasing elements are provided in the form of tab-like elements formed as part of the inner circumference of the one or more support rings, wherein said tab-like elements are caused to fold upwards, downwards (or both upwards and downwards) in relation to the plane of the support ring(s), thereby effectively increasing the vertical height of said ring(s) in these directions. The invention may comprise any suitable number of tab-like elements, preferably two or more.

In a still further preferred embodiment of the present invention, the height-increasing element comprises a tubular fabric sleeve attached to the inner circumference of one or more support rings. In some versions of this embodiment, the fabric sleeve extends from the support ring upwards (i.e. in the direction of the atrium, following implantation). In other versions, said fabric sleeve extends from the support ring in a downwards direction. In still other versions, the fabric sleeve is disposed such that one portion thereof extends upwards, while a second portion extends in a downwards direction. In a further embodiment of this type, two separate fabric sleeves are attached to the inner circumference of the one or more support rings, one extending from the ring(s) upwards, with the other one extending from the ring(s) downwards.

### BRIEF DESCRIPTION OF THE DRAWINGS

FIG. 1 depicts a wave-like wire spring for use as a height-increasing element for a valve support device of the present invention.

FIG. 2 depicts the height-increasing element shown in FIG. 1 after its attachment to the inner circumference of a valve support ring.

FIG. 3 depicts a circular wire spring suitable for use as a height-increasing element for a valve support device of the present invention.

FIG. 4 depicts the height-increasing element of FIG. 3 following its attachment to the inner circumference of a valve support ring.

FIG. 5 shows a single-ring valve support device in its flat, pre-crimped form with comprising four tab-like height increasing elements attached to the inner circumference of the support ring.

### DETAILED DESCRIPTION OF THE PREFERRED EMBODIMENTS

In the context of the present invention, the term “valve support device” is used to refer to any intracardiac device that is adapted for implantation at a cardiac valve annulus (such as—but not limited to—the mitral annulus). The purpose of such devices is to provide a stable platform for the implantation and deployment of a prosthetic valve at said cardiac valve annulus. While the prosthetic valve may be deployed at the same time as valve support device, in many cases (for example, as described in the co-owned, co-pending patent application that published as US 2014/0005778), the prosthetic valve is deployed following implantation of the valve support device, as the second stage in a two-stage procedure. Alternatively the prosthetic valve may be implanted at a different time after the valve support implantation, in a separate procedure, after tissue healing and covering of the valve support device. The second procedure may be, for example, 2-3 months later or even up to several years later.

One of the key advantages of using a valve support device as a platform (rather than directly deploying a prosthetic valve at the annulus) is that this permits the use of standard, commercially-available prosthetic valves, since the stabilization and anchoring within the cardiac tissues becomes a function of the specialized valve support device—rather than necessitating the modification of the prosthetic valve in order to incorporate stabilizing elements. Furthermore, in the case of mitral valve replacement, the valve support device also serves to effectively reduce the diameter of the large mitral valve annulus, thereby facilitating the deployment of a smaller diameter aortic valve within the central space of said support device. In this regard it should be noted that in many (but not all) cases, the valve support device has an annular (or ring-shaped) form, the outer circumference of which engages with the cardiac tissues, while the inner circumference of the “ring” defines a central space into which a prosthetic heart valve may be stably implanted.

In some cases, the cardiac valve support device may comprise a single support ring, as disclosed in co-owned, co-pending WO 2013/128436. In other cases, the support device may comprise two rings mutually connected by bridging elements, as disclosed in co-owned, co-pending US 2014/0005778. However, it is to be recognized that the improved valve compatibility elements of the present invention are also intended for use in conjunction with other forms of cardiac support device not disclosed in these two publications.

As disclosed hereinabove, in one highly preferred embodiment, the cardiac support device is an annular, or ring-shaped, device characterized in having an inner circumference and an outer circumference. As mentioned hereinabove, the intracardiac device of the present invention is suitable for delivery and implantation by the endovascular and/or transapical routes. In this context, the term “is suitable for” refers to the fact that the device is capable of being folded or collapsed (“crimped”) into a low profile, small-diameter conformation that will enable its delivery through a similarly small-diameter delivery catheter or other device. Then, after haven being delivered to the desired implantation site (e.g. the mitral valve annulus), the intracardiac device is released from the confines of the delivery device and allowed to expand into its working conformation. This process of crimping and expansion is, in most embodiments, facilitated by the fact that the intracardiac device is constructed of a shape-memory material such as Nitinol.

In one particularly preferred embodiment the intracardiac device is a cardiac valve support device comprising a single support ring (as disclosed in co-owned WO 2013/128436). Generally, such a valve-support device comprising a single ring-shaped annular support element, has a collapsed delivery configuration and a deployed configuration. In one embodiment, the support element is provided in the form of flat annular ring, preferably constructed from a material having superelastic and/or shape memory properties. One example of such a suitable material is Nitinol, which possesses both of the aforementioned physical properties. These properties may be utilized in order to permit said device, following its delivery in a collapsed conformation, to return to an expanded memory configuration after being heated above its transition temperature. In the radial plane (i.e. the plane in which the native cardiac valve leaflets are disposed when in their closed position), the size of the annular support element may be defined in terms of its outer radius (Ro), its inner radius (Ri) and the difference between these two radii (Rd). It should be appreciated that Ro is determined by the diameter of the mitral valve annulus into which the valve support device will be implanted. Ri, however, is determined by the outer diameter of the replacement heart valve that will be inserted into the central space of the support device. Generally, the prosthetic aortic valves used in conjunction with the valve support device of the present invention have an external diameter considerably less than that of the mitral valve annulus. It may therefore be appreciated that Rd approximately corresponds to the annular gap between the small outside-diameter replacement valve and the relatively large diameter mitral valve annulus. Preferably, Rd is in the range of 1-14 mm. In most embodiments of the valve support device of the present invention, the inner radius of the single-ring support element is in the range of 23-29 mm and the outer radius thereof is in the range of 30-50 mm.

The above-described single-ring valve support device is particularly suitable for two-step endovascular and/or transapical implantation procedures for replacing a patient's native mitral valve. In general, the support structure is first delivered in a collapsed conformation within a delivery device and positioned near or within a mitral valve annulus and secured in place. A replacement cardiac valve is subsequently secured to the support structure, securing the replacement valve in place near or within the annulus. By implanting the support structure and replacement cardiac valve in two steps, the replacement valve can have a lower delivery profile because it does not have to expand as much to contact native tissue due to the presence of the support structure. This eliminates the need to have a large delivery profile replacement valve as would be required if attempting to position a mitral valve at the native mitral valve annulus, or if attempting to position a one-piece mitral valve implant (i.e., an implant not assembled in-vivo) within the native mitral valve. Examples of suitable delivery systems that may be used to implant the single-ring valve support device of the present invention are disclosed in co-owned WO 2013/128436 and WO 2014/128705, the contents of both of which are incorporated herein in their entirety.

In one preferred embodiment, said valve support device is suitable in size and shape for implantation at the mitral valve annulus. In another preferred embodiment, the valve support device is suitable in size and shape for implantation within or adjacent to the aortic valve. With regard to the size of valve support devices suitable for implantation at these two anatomical sites, support devices intended for use at the mitral valve annulus will generally have an external radius in the range of 25 to 55 mm, while support devices intended for use at the aortic valve annulus will generally have an external radius in the range of 20 to 35 mm.

The valve support device of the present invention may be used to provide a stable implantation platform for any suitable prosthetic cardiac valve. As explained hereinabove, the valve support device of the present invention may be used to effectively narrow the diameter of the mitral valve annulus, thereby enabling the subsequent implantation of one of the smaller commercially-available replacement aortic valves. Thus, in one particularly preferred embodiment, the prosthetic valve implanted within the central space of the valve support device is a prosthetic aortic valve. Non-limiting examples of such prosthetic valves include the Lotus valve (Boston Scientific, Marlborough, Mass.) and the Sapien valve (Edwards Lifesciences Corp., Irvine, Calif.).

In the case that the height-increasing components are spring elements, these may be manufactured separately from the device, and then attached thereto at the desired location with the aid of surgical sutures or other mechanical form of attachment, within a biocompatible fabric covering the device. Furthermore, in some embodiments, said spring elements may be covered with biocompatible plastic or polymer, for example a silicone tube.

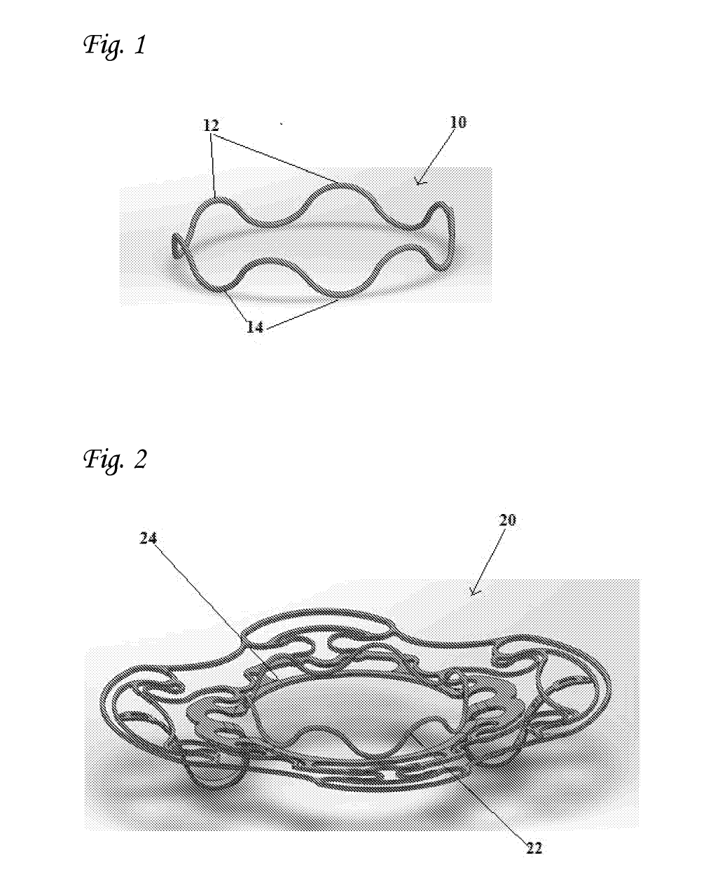

In one preferred version of this embodiment of the invention, the wire spring is constructed in the form of a wavy wire (for example, having the approximate shape of a sinusoidal wave) having its two ends joined together, thereby causing said spring to adopt the approximate shape and form of a closed loop (similar to a common type of bed-spring). An example of such a wavy spring loop is depicted in FIG. 1. In this figure, spring 10 is seen to comprise an upper border (formed by an imaginary line joining the highest portions 12 of said spring, and a lower border, formed by an imaginary line joining the lowest portions 14 thereof.

FIG. 2, depicts a single-ring valve support device 20 in which a single wavy spring loop 22 (as shown in FIG. 1) is attached at its lower border to the inner circumference of support ring 24. As may be seen in this figure, this type of attachment leads to an increase in the effective height of the support ring in an upwards direction (i.e. towards the atrium).

In one preferred version of this embodiment of the invention, the wire spring is constructed in the form of a non-wavy, hoop-like circle, which may be attached either to the upper face of the valve support ring, adjacent to the inner circumference thereof (thereby increasing the height of said ring in an upward direction), or to the lower face thereof (thereby increasing the height of said ring in a lower direction). Two or more such wires may be used to increase the height of said ring even further, and may be attached to the upper face of the valve support ring, the lower face, or both.

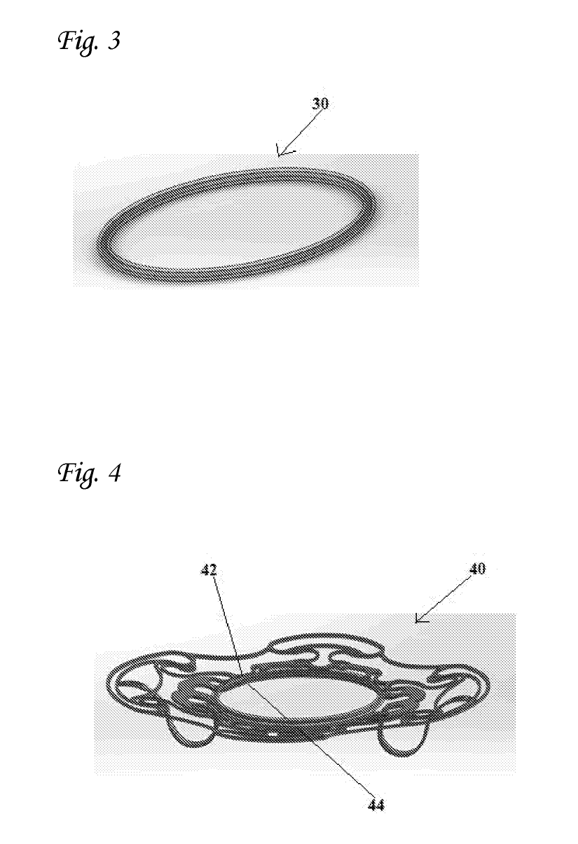

An example of this type of wire hoop spring is shown (30) in FIG. 3.

FIG. 4 illustrates a single-ring valve support device 40, in which a wire hoop spring 42 (of the same design as shown in FIG. 3) is attached to the upper face of support ring 44, close to the inner circumference thereof.

As disclosed hereinabove, in some embodiments of the present invention, the height-increasing elements comprise one or more tab-like elements formed as part of the inner circumference of the one or more support rings, wherein said tab-like elements are caused to fold upwards, downwards (or both upwards and downwards) in relation to the plane of the support ring(s), thereby effectively increasing the vertical height of said ring(s) in these directions. Preferably, the device comprises two or more height-increasing tabs.

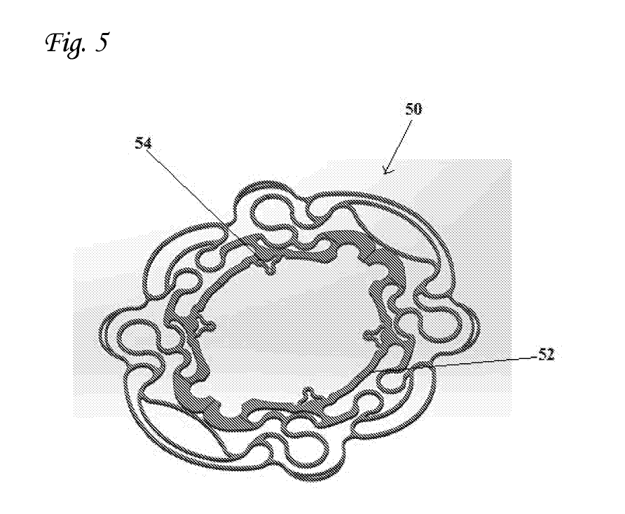

FIG. 5 illustrates a single-ring valve support device 50 of the present invention in its flat, pre-folded form, in which the inner circumference of the support ring 52 includes four tab-like elements 54. As part of the stage of manufacturing and shaping of this flat-form of the device, the tab-like elements will be caused to be folded and fixed (by means of heat setting) in the desired directions (upwards, downwards, or both).

In all embodiments of the present invention, the entire device (including the additional height-increasing elements above, below, or both above and below the inner border of the support ring) may be covered with a fabric (such as PTFE, Dacron, Polyester or other biocompatible material). When covered in this way, the shape of the internal border of the support ring is essentially transferred from being a flat ring shape to a 3 dimensional tube-like element, having a longitudinal length (height) of approximately 3-20 mm, and a thickness of 0.2-1 mm.

In the embodiments in which the height-increasing element is provided in the form of a fabric sleeve having a generally-tubular shape, said sleeve may be constructed from any suitable biocompatible fabric, including, but not limited to PTFE, Dacron or Polyester. In some cases, the fabric sleeve will be constructed of a single type of fabric. In other embodiments, the sleeve may comprise two or more different fabric materials. As in the case of the fabric covered spring elements described above, the fabric sleeve preferably has a height in the range of 3-20 mm and a thickness of 0.2-1 mm. In some embodiments, the fabric sleeve may be constructed such that it has a uniform thickness, while in other embodiments the sleeve is constructed of two or more different fabric portions, each having a different thickness.

The valve support device of the present invention is preferably constructed of Nitinol wire or sheet, and/or any other suitable biocompatible shape memory material.

1. A valve support device suitable for endovascular and/or transapical implantation at or near to a cardiac valve annulus, comprising one or more support rings and one or more height-increasing elements attached to the inner circumference of said support rings.

2. The valve support device according to claim 1, wherein the height-increasing element is a wire spring.

3. The valve support device according to claim 2, wherein the wire spring is covered with a biocompatible fabric.

4. The valve support device according to claim 1, wherein the height-increasing element is a tubular sleeve constructed from a biocompatible fabric.

5. The valve support device according to claim 1, wherein the height-increasing element is a tab-like element.

6. The valve support device according to claim 1, wherein the one or more height-increasing elements are located above the plane of the support ring.

7. The valve support device according to claim 1, wherein the one or more height-increasing elements are located below the plane of the support ring.

8. The valve support device according to claim 1, wherein the one or more height-increasing elements are located both above and below the plane of the support ring.

9. The valve support device according to claim 1, wherein the height-increasing elements have a vertical height in the range of 3-20 mm.

10. The valve support device according to claim 1, wherein said device is suitable in size and shape for implantation at the mitral valve annulus.
